(19) United States
(12) Patent Application Publication
(10) Pub. No.: US 2011/0022174 A1
(43) Pub. Date: Jan. 27, 2011

(54) MODELING MICRO-SCAFFOLD-BASED IMPLANTS FOR BONE TISSUE ENGINEERING

(75) Inventors: Yaron Holdstein, Haifa (IL); Anat Fischer, Haifa (IL)

Correspondence Address: Browdy and Neimark, PLLC, 1625 K Street, N.W., Suite 1100, Washington, DC, 20006, US

(73) Assignee: TECHNION - RESEARCH & DEVELOPMENT FOUNDATION LTD, HAIFA (IL)

(21) Appl. No.: 12/812,283

(22) PCT Filed: Jan. 11, 2009
(86) PCT No.: PCT/IL09/00044

§ 371 Date: Oct. 4, 2010

Related U.S. Application Data
(60) Provisional application No. 61/020,567, filed on Jan. 11, 2008.

(51) Int. Cl. A61F 2/28; G06F 17/50; G06K 9/00

(52) U.S. Cl. 623/16.11; 703/1; 382/128

(57) ABSTRACT

A new conceptual biomedical method is presented for designing scaffold-based bone implants and using these implants in treating deteriorated bones. These implants have micro-architectural bone structures that are capable of mimicking the stochastic micro-structure as in natural bone bio-mineral structures. Moreover, they can be adapted as specific tailor-made compatible bone-repair mediator implants to be used as effective substitutes for natural damaged bone fracture structures.

### FIELD OF THE INVENTION

The present invention relates to biomedical methods for designing scaffold-based implants. More particularly, the present invention relates to the design of implants for treating damaged bones.

### BACKGROUND OF THE INVENTION

Currently, the biomedical community is very interested in creating and developing scaffolds to be used as a base for bone micro-implants. Diseases such as osteoporosis are characterized by increased bone fragility, which leads to micro-architectural deterioration of bone tissue and eventually to micro-fractures.

At the micro-structural level, bone is constructed from thin rods, known as trabeculae, and plates. These rods and plates are arranged in semi-regular, three-dimensional patterns and constitute highly anisotropic and heterogenic material. Recent state-of-the-art methods for diagnosing bone fractures rely on emerging technology and advanced methods for 3D micro volumetric scanning, modeling and analyzing bone micro-structure. This structure is known to be stochastic in nature and varies for each diagnosed bone fracture, depending upon the following main parameters: patient, bone type, location, and type of fracture of a specific bone. FIGS. 1a and 1b depict two views of bone growth over a scaffold (state of the art). The figures show bone tissues growing over an implant that serves as a base for healing the fractured bones. Modeling, designing, engineering, and installing a bone implant to form this type of specific scaffold surface offer major advantages for the healing process.

The assumption is that the resulting bone structure will resemble the scaffold structure. Therefore, from the point of view of healing functionality, the shape of the scaffold-based implant should be designed to mimic the natural bone structure as closely as possible.

Currently, state-of-the-art research in the field of tissue engineering focuses primarily on selecting materials that can be absorbed by the bone as well as on bio-manufacturing schemes for creating scaffolds that preserve the strength of the bone structure. The shape and topology of the scaffold structures have porous layouts. Since current structures are marked by a simple, symmetric and standard layout, they cannot be customized to a specific bone type or to a specific patient treatment case. However, each individual type of bone fracture varies from one medical case to another, as does the local position of the fracture within that bone. Current state-of-the-art methods pay no attention to variations in scaffold structures and do not supply the optimal inherent mechanical structure.

Indeed, today there is a big gap between the standardized structure and the optimal customized structure for bone implant production. Standardized structures do not selectively discriminate between micro-structural architecture in individual fractures of a given bone and therefore do not constitute the optimal structure for the healing process. This conceptual gap probably evolved from the belief that current technology is limited and can only provide a generalized solution for designing bone scaffold-based implants. Nevertheless, more specific and complex methods must be developed for designing and modeling more specific structures for the complex problem described herein.

### SUMMARY OF THE INVENTION

One objective of the present invention is to provide a method for generating implants and scaffolds that substantially resemble the micro-structural architecture of the natural bone by introducing more advanced and complex stochastic imaging and modeling. Moreover, in accordance with one aspect of the method used in the present invention, customized scaffold-based implants that vary in location, size and shape can be designed. This customization is achieved by applying a 3D stochastic texture synthesis on the damaged bone model.

Another objective of the present invention is to provide a new image modeling system for designing bone scaffold-based implants that significantly improve the healing process. Since the newly designed bone implants mimic the micro-structure architecture, the resulting bone better integrates into its surroundings. Moreover, the implanted bone connects smoothly to the specific fractured bone according to topological and geometrical characteristics. Therefore, it provides a seamless sub-mesh that fits the scaffold-based implant into the surroundings of a cavity, so that the implant/scaffold functions better than bone grown on a standard scaffold.

Yet another objective of the present invention and the embodiments thereof is to preserve the global mechanical micro-structure of the bone. This is achieved by applying a topological optimization of the mechanical properties of the scaffold-based bone implant with respect to the neighborhood of the damaged region of the bone.

It is therefore provided in accordance with a preferred embodiment of the present invention a method for modeling micro-scaffold-based implants for engineering of bone tissue comprising:
identifying stochastic sampled pattern in the bone tissue;
developing 3D synthesis for said stochastic sampled pattern so as to establish an implant model;
optimizing said implant model based on mechanical constraints;
merging said implant model with the bone tissue.

Furthermore and in accordance with another preferred embodiment of the present invention, the method further comprises:
detecting 3D holes in the bone tissue.

Furthermore and in accordance with another preferred embodiment of the present invention, detecting 3D holes comprises:
scanning medical model of the bone tissue;
extracting 3D micro-structure image from the bone tissue.

Furthermore and in accordance with another preferred embodiment of the present invention, said developing 3D synthesis for said stochastic sampled pattern comprises:
reconstructing a 3D triangular mesh image out of a set of slice-by-slice 2D digitized images;
analyzing said 3D triangular mesh image to evaluate the quality of the mesh;
extracting 3D holes from said 3D triangular mesh image;
determining 3D sampled patterns based on geometric criteria and topological criteria of surroundings of said 3D holes;
adaptively fitting said 3D sampled patterns to said 3D holes;
optimizing a texture by mechanical criteria exerted on said bone tissue.

Furthermore and in accordance with another preferred embodiment of the present invention, said extracting 3D holes from said 3D triangular mesh image comprises identifying cavities representing said 3D holes by setting up a predetermined size threshold wherein a size of each of said 3D holes is compared to said predetermined size threshold and wherein if a volume of a cavity of said cavities is larger than said predetermined size threshold, said cavity is defined as a hole.

Furthermore and in accordance with another preferred embodiment of the present invention, said adaptively fitting said 3D sampled patterns to said 3D holes comprises seamlessly in-filling said 3D holes according to said 3D sampled patterns so that a resulting mesh appears as a single continuous 3D structure.

Furthermore and in accordance with another preferred embodiment of the present invention, said determining 3D sampled patterns comprises searching an appropriate pattern in said 3D triangular mesh image wherein said appropriate pattern best fits the 3D hole according to geometric analysis both on said appropriate pattern and on the 3D hole.

Furthermore and in accordance with another preferred embodiment of the present invention, said determining 3D sampled patterns comprises searching an appropriate pattern in said 3D triangular mesh image wherein said appropriate pattern best fits the 3D hole according to topological analysis both on said appropriate pattern and on the 3D hole.

Furthermore and in accordance with another preferred embodiment of the present invention, said developing 3D synthesis comprises for each voxel in a synthesized mesh:
getting a cubic region Regᵢ from the synthesized mesh, centered at Vᵢ;
getting a cubic region Regⱼ from a sample mesh, centered at Vⱼ;
defining a measured distance dᵢⱼ, between Regᵢ and Regⱼ such that dᵢⱼ=d(Regᵢ, Regⱼ);
creating a set {Regⱼ} having a good correlation with Regᵢ;
selecting Regⱼ with a highest correlation from the set {Regⱼ} and assigning its center voxel value Vⱼ to the voxel Vᵢ in the synthesized mesh.

Furthermore and in accordance with another preferred embodiment of the present invention, the method further comprises integrating a mask containing geometric features in high contrast.

Furthermore and in accordance with another preferred embodiment of the present invention, said developing 3D synthesis comprises for each voxel in a synthesized mesh wherein an input is a cubic region Regᵢ from the synthesized mesh that defines a hole, centered at Vᵢ:
getting a cubic region Regⱼ from the sample mesh, centered at Vⱼ;
defining a measure distance dᵢⱼ, between Regᵢ and Regⱼ, such that dᵢⱼ=d(Regᵢ, Regⱼ);
create a set {Regⱼ} having good correlation with Regᵢ;
selecting Regⱼ with the highest correlation from the set {Regⱼ};
copying {Regⱼ} to the synthesized mesh.

Furthermore and in accordance with another preferred embodiment of the present invention, the method further comprises adding parameters characterizing the bone.

Furthermore and in accordance with another preferred embodiment of the present invention, said parameters are selected from a group of parameters such as bone density and directionality.

It is yet provided in accordance with yet another preferred embodiment of the present invention, a reconstructed implant to be filled within a hole in a fractured or diseased bone that is designed according to the method described herein before.

Furthermore and in accordance with another preferred embodiment of the present invention, wherein said implant fills in the hole in a seamless manner.

### BRIEF DESCRIPTION OF THE FIGURES

FIG. 1 shows two views ((a) and (b)) of bone growth over a scaffold (prior art).

FIG. 2 depicts a block diagram of steps in implementing a method for generating a 3D scaffold-based implant in accordance with a preferred embodiment of the present invention.

FIG. 3 illustrates analyses of: (a) original model, with (b) artificially generated hole, (c) micro-structure 3D texture synthesized in accordance with a preferred embodiment of the present invention, and (d) symmetric scaffold, where R is approximately 10 units (˜350μ).

### DETAILED DESCRIPTION OF THE INVENTION

Some embodiments of the invention are herein described, by way of example only, with reference to the accompanying drawings. With specific reference to the drawings in detail, it is stressed that the particulars shown are by way of example and for purposes of illustrative discussion of the preferred embodiments of the present invention only. These particulars are presented for the purpose of providing what is believed to be the most useful and readily understood description of the principles and conceptual aspects of the invention. In this regard, no attempt is made to show structural details of the invention in more detail than is necessary for a fundamental understanding of the invention. The description, together with the drawings, makes it apparent to those skilled in the art how the several forms of the invention may be embodied in practice.

The scaffold-based implant structure is defined by applying volumetric hole in-filling to diseased cavities of the bone micro-structure. These scaffold-based implants can be designed and produced in advance before they are used.

Some embodiments of the present invention offer new and unique methods for detecting and characterizing damaged cavities by applying a 3D imaging technique before hole in-filling. Diseased bone cavities are not straightforward due to the porous and deformed nature of the bone micro-structure.

Some embodiments of the present invention use a 3D computational modeling method that is based on a 3D texture synthesis technique. This method is an extension of the 2D method to three dimensions to achieve the desired outcome of volumetric micro-cavity hole in-filling. This new method can be used to reproduce the micro-structural architecture in a sample of fractured bone, thus providing the designer and engineer with a bone scaffold-based implant required for better bone growth and improved healing. Two modeling methods have been extended and implemented: voxel-by-voxel and block-wise texture syntheses. In the 3D images produced by computational modeling, the resulting topology is much more complex than in the 2D case. Moreover, the bone has a 3D stochastic texture structure which has no exact pattern repetitions.

Some embodiments of the present invention present a novel method for modeling natural scaffold-based implants to fill in cavities (holes) in cancellous bone caused by bone diseases. As mentioned herein before, this type of bone is characterized by a complex micro-structure composed mainly of trabecula modeled as thin cylindrical rods and plates. Cavities in the 3D micro-structure are identified by measuring the cavity volumes and comparing them to a specified threshold. The present invention provides a novel method for seamless in-filling of these holes using deformed elements consistent with the 3D neighborhood of a given hole, and therefore provides highly improved implants. The hole in-filling is based on a 3D pattern-growing scheme, a 3D texture synthesis that takes into account the exerted forces so that the global directionality of the micro-structure is preserved. Furthermore, another goal of some embodiments of the present invention is to optimize the scaffold according to the mechanical properties of the bone. This scheme can take the exerted forces into account so that the global directionality of the micro-structure is preserved.

A main contribution of this invention is the development of customized micro-implants according to given bone micro-structures.

Some embodiments of the present invention describe a novel method for modeling scaffold-based implants that have the stochastic structure of bone and can be customized according to given bone structures. The method for designing these implants is based on applying a 3D texture synthesis technique that can create a scaffold to be inserted to the damaged cavities of a given bone. These scaffold-based implants can replace the diseased cavities in the cancellous bone.

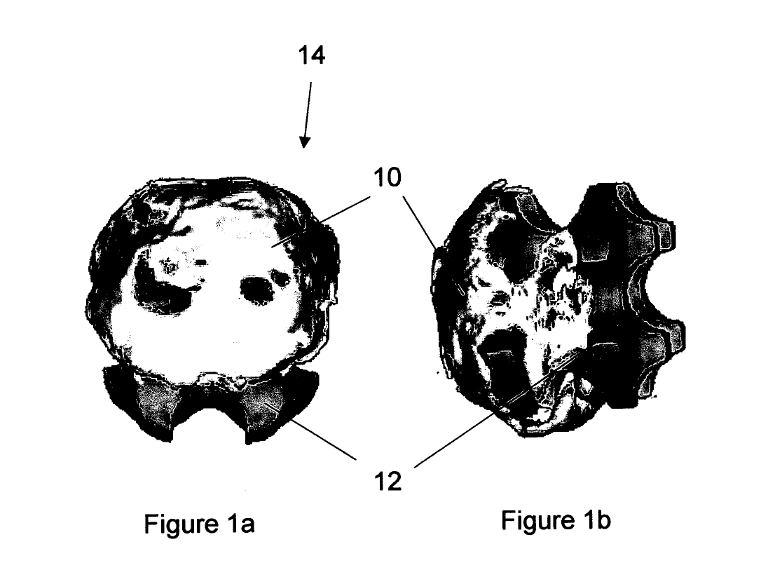

Reference is made to FIGS. 1a and 1b, depicting two views of bone growth over a scaffold (prior art). In the figures, bone tissues grow over a standard implant that forms a scaffold for healing fractured bones. One of the main features of the present invention is a structure called a scaffold that is placed onto the bone so the bone can grow in areas where its micro-structure has been corrupted. The scaffold has two purposes: (a) its structure facilitates the growth of the bone around it, and (b) the material forming the scaffold is consumed by the bone and eventually degrades over the years as new healthy bone replaces the scaffold material. The inventors of the present invention have shown that the resulting bone structure resembles the scaffold structure; therefore, its shape is critical from the point of view of functionality.

Implementations of the present invention comprise the following steps:
Detecting 3D holes in the cancellous bone sample.
Developing 3D texture synthesis for stochastic pattern of bone scaffold-based implants.
Developing topology optimization of the implants, based on mechanical constraints.
Merging the scaffold-based implant with its bone local neighborhood.

The present invention has evolved a process for 3D in-filling of holes in a volumetric micro-structure. Some implementations of the present invention work on deformed 3D volumetric bone textures, such as the trabecular bone micro-structure, rather than on segments and their topological relations. The present invention shows that the texture synthesis approach is more natural for bone micro-structures.

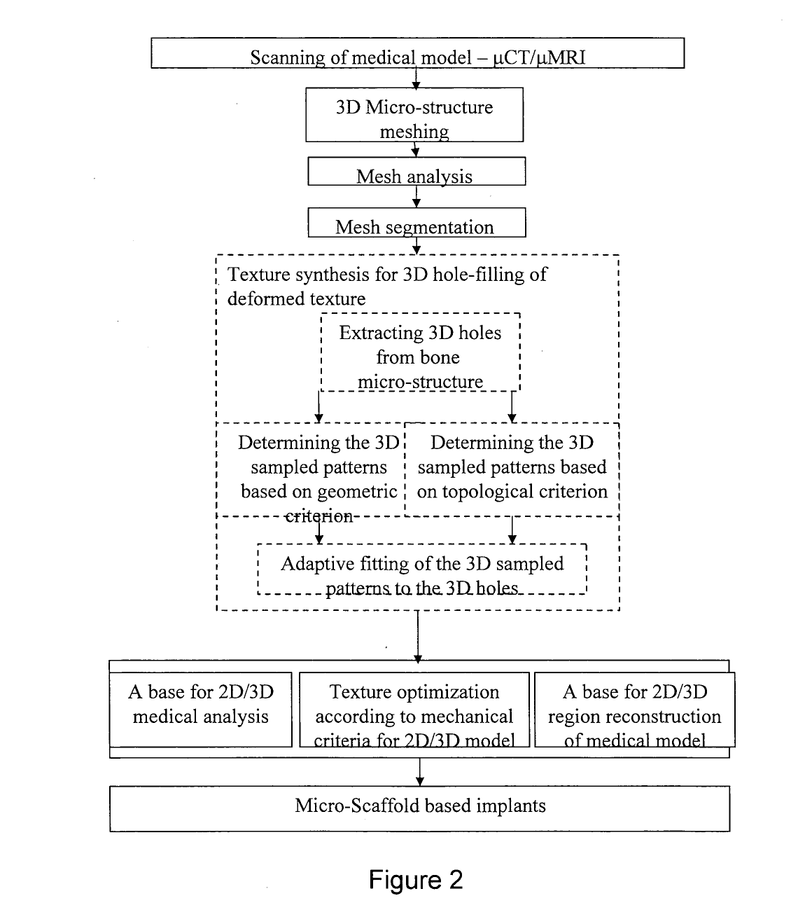

Reference is now made to FIG. 2, which shows a block diagram of steps taken in implementing the method of generating a scaffold in accordance with a referred embodiment of the present invention. The method involves scanning a medical model from μCT/μMRI and extracting its micro-structure 3D image (3D computerized model). Initially, the medical condition of the bone fractures is acquired either from μCT or μMRI images, where the input is digitized slice by slice, with each slice constituting a 2D image. A 3D model is extracted from the set of 2D slices, and 3D diagnostic methods are then applied.
3D texture synthesis is performed for 3D hole-filling of the deformed texture. The development of the method includes the following operations:
3D micro-structure meshing—Reconstructing a 3D triangular mesh out of the set of 2D images.
Mesh analysis—Performing mesh analysis to evaluate the quality of the mesh.
Extracting 3D holes from bone micro-structure—Identifying the cavities representing the holes. This operation is not straightforward, since the structure of the bone is cancellous and is thus characterized by many hole-like patterns, forming a model of high genus. The criterion is size. If the volume of a cavity is larger than a certain threshold, it is defined as a hole.
Determining the 3D sampled patterns based on geometric and topological criteria—Analyzing the surroundings of the hole and selecting a sample pattern that best fits the hole identified. This analysis takes both geometric and topological aspects into consideration.
Adaptive fitting of 3D sampled patterns to the 3D holes—Applying a 3D texture synthesis-based method for bone structure. This method is based on seamlessly in-filling the hole according to the sample, so that the resulting mesh will appear as a single continuous 3D structure.
Texture optimization—Texture optimization according to mechanical criteria.
   Image in-Filling and Hole Patching:

The present invention makes use of a voxel-by-voxel approach since this approach has more degrees of freedom when choosing a new pixel value. Optionally, a patch-wise approach can be used which preserves the bone features better.

### Analysis:

Texture synthesis for 3D hole in-filling is described as a texture synthesis process in 3D space. The following operations are needed:

### Extracting 3D Holes of Bone Micro Structure:

In this stage, a volumetric cavity in the mesh that represents a hole in the bone structure is identified. This volume is characterized by sparse and relatively thin trabeculae.

### Determining the 3D Sampled Patterns Based on Geometric Criteria:

In this stage, an appropriate pattern in the mesh is searched, wherein this pattern best fits the hole found in the previous stage. The match of such a pattern is determined by applying geometric analysis both on the pattern and on the hole.

### Determining the 3D Sampled Patterns Based on Topological Criteria:

In this stage, an appropriate pattern in the mesh is searched, wherein this pattern fits the hole previously found. Here, the match of the pattern is determined by applying topological analysis both on the pattern and on the hole.

### Adaptive Fitting of the 3D Sampled Patterns to the 3D Holes:

In this stage, holes are filled in using samples found in the prior two stages. The hole is filled in by applying a volumetric texture synthesis scheme, given the volume to be filled and the sample pattern. This is performed under the assumption that the bone pattern containing the hole also has regions with a normal, uncorrupted structure.

Moreover, in certain cases where features in the resulting model should be preserved, a feature extraction method for hinting at and assisting the growth process can be applied, as for example in Lefebvre, et al., 2006.

The algorithms used in the preferred implementation of the present invention:

The algorithm applied in the preferred implementation of the present invention is based on the pixel-by-pixel approach introduced by Efros, et al., 1999. The following steps illustrate a 3D extension of the 2D case:

For each voxel Vᵢ in the synthesized mesh:
Get a cubic region Regᵢ from the synthesized mesh, centered at Vᵢ.
   This region may contain original voxels taken from the sample, synthesized ones and invalid ones (that were not initialized).
Get a cubic region Regⱼ from the sample mesh, centered at Vⱼ.
Define a distance measure dᵢⱼ, between Regᵢ and Regⱼ, such that dᵢⱼ=d(Regᵢ, Regⱼ).
Create the set {Regⱼ} that has a good correlation with Reg.
Select Regⱼ with the highest correlation from the set {Regⱼ} and assign its center voxel value Vⱼ to the voxel Vᵢ in the synthesized mesh.

Optionally, the synthesized patterns are improved by integrating a mask that contains the main geometric features and shape characteristic in high contrast. This integration can be implemented via a weighting process. Creating the features mask can optionally involve segmentation and feature detection.

Optionally, 3D texture synthesis based on the patch-wise approach (block by block) can be applied. The stages are described as follows:

For each voxel Vᵢ in the synthesized mesh:
The input: A cubic region Regᵢ from the synthesized mesh that defines a hole, centered at Vᵢ. This region can contain original voxels taken from the sample, synthesized ones and invalid ones (that were not initialized).
Get a cubic region Regⱼ from the sample mesh (the sampled window), centered at Vⱼ.
Define a distance measure dᵢⱼ, between Regᵢ and Regⱼ, such that dᵢⱼ=d(Regᵢ, Regⱼ).
Create the set {Regⱼ} that has a good correlation with Regᵢ.
Select Regⱼ with the highest correlation from the set {Regⱼ} and copy it entirely to the synthesized mesh.

Optionally, this approach can be improved by introducing a minimal cut optimization scheme between adjacent patches.

Optionally, the method of the present invention as described herein can be improved by adding parameters of bone density and directionality. Thus, a block can be added according to shape correlation and density threshold and according to the directionality of the surroundings volume of that block.

### Criteria Evaluation:

Following are the criteria for evaluating the results of the invention:
Evaluation of the correlation between the sample and the 3D synthesized meshes.
Evaluation of the correlation between an average 3D pattern in the sample and the 3D synthesized mesh.
Comparison of the averaged area/volume of the holes within a selected region in the sample, assuming the region is the same size as the sample image.
Comparison of the number of holes within a selected region (density of holes) with the number of holes in the sample, assuming the region is the same size as the sample image.

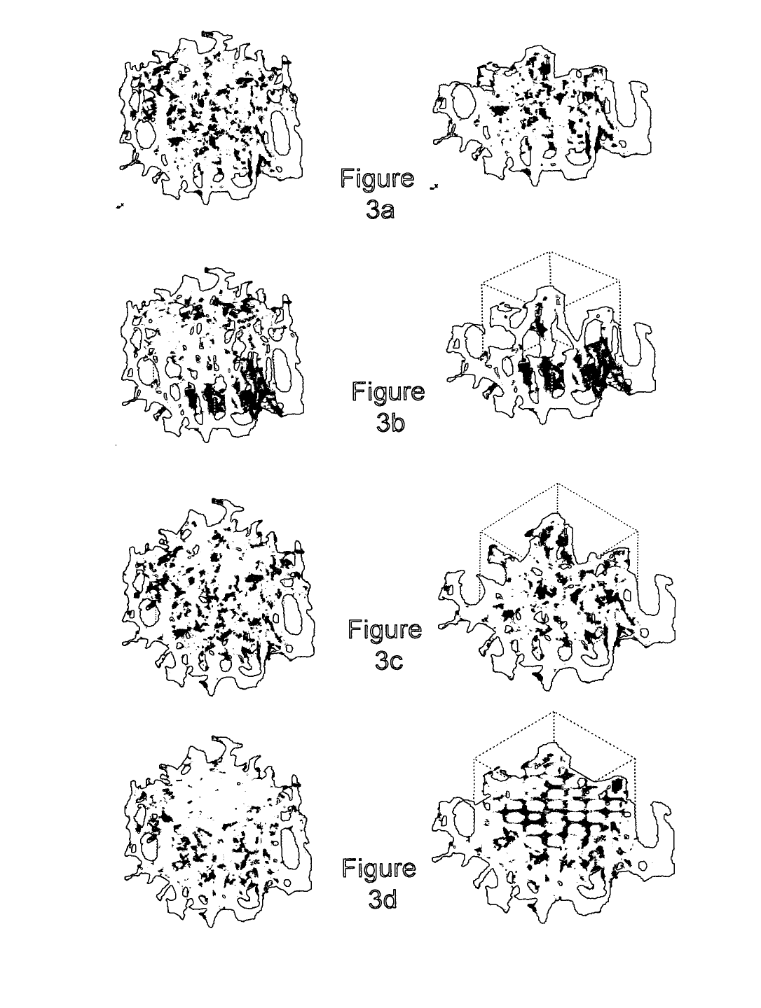

Reference is now made to FIG. 3 depicting the following analysis: (a) original model, with (b) artificially generated hole, (c) micro-structure synthesized in accordance with a preferred embodiment of the present invention and (d) symmetric scaffold where R is approximately 10 units (˜350μ).

The results illustrate the reconstruction of a bone that models a scaffold-based implant using the proposed 3D texture synthesis method. The results are compared with other bone models that were filled in by standard scaffold-based implants. The stresses acting upon the structure are illustrated, with the entire implant illustrated on the left side and a cross-sectional view of the implant shown for clarity purposes on the right side. The color scaling ranges from blue (minimal stress) through green (average stresses) up to red (maximal stresses). As seen in the figure, the stress distribution for the micro-structure synthesized model of the proposed method is almost the same as for the original sample of the healthy bone structure. Moreover, the standard scaffold-based implant bears minimal stress distribution, reducing the risk that the surrounding healthy bone structure might be harmed due to incorrect load exertion to it.

### REFERENCES

Almeida H. A., Bartolo P. J. and Ferreira J. C. Mechanical behavior and vascularisation of tissue engineering scaffolds [Conference]//3rd International Conference on Advanced Research in Virtual and Rapid Prototyping.—2008.
Bhat Pravin, Ingram Stephen and Turk Greg Geometric texture synthesis by example [Conference]//SGP '04: Proceedings of the 2004 Eurographics/ACM SIGGRAPH symposium on Geometry processing.—Nice: ACM Press, 2004.-pp. 41-44.
Chen G., Ushida T. and Tateishi T. Scaffold Design for Tissue Engineering [Journal]//Macromolecular Bioscience.—2002.-2: Vol. 2.-pp. 67-77.
De-Bonet Jeremy S. Multiresolution sampling procedure for analysis and synthesis of texture images [Conference]//SIGGRAPH '97: Proceedings of the 24th annual conference on Computer graphics and interactive techniques.—[s.l.]: ACM Press/Addison-Wesley Publishing Co., 1997.-pp. 361-368.
Efros Alexei A. and Freeman William T. Image Quilting for Texture Synthesis and Transfer [Journal]//Proceedings of SIGGRAPH 2001.—[s.l.]: ACM Press/ACM SIGGRAPH, 2001.-pp. 341-346.
Efros Alexei A. and Leung Thomas K. Texture Synthesis by Non-parametric Sampling [Conference]//IEEE International Conference on Computer Vision.—1999.-pp. 1033-1038.
Heeger David J. and Bergen James R. Pyramid-based texture analysis/synthesis [Conference]//SIGGRAPH '95: Proceedings of the 22nd annual conference on Computer graphics and interactive techniques.—[s.l.]: ACM Press, 1995.-pp. 229-238.
Holdstein Y. and Fischer A. 3D Surface Reconstruction for bone micro-structures using Meshing Growing Neural Gas (MGNG) [Conference]//International Conference on “Advanced Research in Virtual and Rapid Prototyping (VRAP)”.—Leiria: [s.n.], 2007.
Holdstein Y. and Fischer A. Reconstruction of Volumetric Freeform Objects using Neural Networks [Conference]//The 7th Korea-Israel Bi-National Conference on Geometrical Modeling and Computer Graphics.—Seoul: [s.n.], 2006.
Lagae Ares, Dumont Olivier and Dutre Philip Geometry Synthesis by Example [Conference]//SMI '05: Proceedings of the International Conference on Shape Modeling and Applications 2005.—[s.l.]: IEEE Computer Society, 2005.-pp. 176-185.
Rho J. Y., Kuhn-Spearing L. and Zioupos P. Mechanical properties and the hierarchical structure of bone [Journal]//Medical Engineering & Physics.—1998.-Vol. 20.-pp. 92-102.
Wei Li-Yi and Levoy Marc Fast texture synthesis using tree-structured vector quantization [Conference]//SIGGRAPH '00: Proceedings of the 27th annual conference on Computer graphics and interactive techniques.—[s.l.]: ACM Press/Addison-Wesley Publishing Co., 2000.-pp. 479-488.
Zhou Kun [et al.] Mesh quilting for geometric texture synthesis [Journal]//ACM Transactions on Graphics (TOG).—[s.l.]: ACM Press, 2006.-3: Vol. 25.-pp. 690-697.
L. Podshivalov, Y. Holdstein, A. Fischer, P. Z. Bar-Yoseph, “Towards a multi-scale computerized bone diagnostic system: 2D micro-scale finite element analysis”, Journal of Communications in Numerical Methods in Engineering, Accepted, November 2008.
Y. Holdstein, L. Podshivalov, A. Fischer, P. Z. Bar-yoseph, “A Neural Network Technique for Re-meshing of Bone Micro-Structure”, International Journal of Shape Modeling (IJSM), Special Issue, Accepted, June 2008.

1. A method for modeling micro-scaffold-based implants for engineering of bone tissue comprising:
identifying stochastic sampled pattern in the bone tissue;
developing 3D synthesis for said stochastic sampled pattern so as to establish an implant model;
optimizing said implant model based on mechanical constraints;
merging said implant model with the bone tissue.

2. The method as claimed in claim 1, further comprising: detecting 3D holes in the bone tissue.

3. A method as claimed in claim 2, wherein detecting 3D holes comprises:
scanning medical model of the bone tissue;
extracting 3D micro-structure image from the bone tissue.

4. A method as claimed in claim 1, wherein said developing 3D synthesis for said stochastic sampled pattern comprising:
reconstructing a 3D triangular mesh image out of a set of slice-by-slice 2D digitized images;
analyzing said 3D triangular mesh image to evaluate the quality of the mesh;
extracting 3D holes from said 3D triangular mesh image;
determining 3D sampled patterns based on geometric criteria and topological criteria of surroundings of said 3D holes;
adaptively fitting said 3D sampled patterns to said 3D holes;
optimizing a texture by mechanical criteria exerted on said bone tissue.

5. The method as claimed in claim 4, wherein said extracting 3D holes from said 3D triangular mesh image comprises identifying cavities representing said 3D holes by setting up a predetermined size threshold wherein a size of each of said 3D holes is compared to said predetermined size threshold and wherein if a volume of a cavity of said cavities is larger than said predetermined size threshold, said cavity is defined as a hole.

6. The method as claimed in claim 4, wherein said adaptively fitting said 3D sampled patterns to said 3D holes comprises seamlessly in-filling said 3D holes according to said 3D sampled patterns so that a resulting mesh appears as a single continuous 3D structure.

7. The method as claimed in claim 4, wherein said determining 3D sampled patterns comprises searching an appropriate pattern in said 3D triangular mesh image wherein said appropriate pattern best fits the 3D hole according to geometric analysis both on said appropriate pattern and on the 3D hole.

8. The method as claimed in claim 4, wherein said determining 3D sampled patterns comprises searching an appropriate pattern in said 3D triangular mesh image wherein said appropriate pattern best fits the 3D hole according to topological analysis both on said appropriate pattern and on the 3D hole.

9. The method as claimed in claim 1, wherein said developing 3D synthesis comprises for each voxel in a synthesized mesh:
getting a cubic region Regᵢ from the synthesized mesh, centered at Vᵢ;
getting a cubic region Regⱼ from a sample mesh, centered at Vⱼ;
defining a measured distance dᵢⱼ, between Regᵢ and Regⱼ, such that dᵢⱼ=d(Regᵢ, Regⱼ);
creating a set {Regⱼ} having a good correlation with Regᵢ;
selecting Regⱼ with a highest correlation from the set {Regⱼ} and
assigning its center voxel value Vⱼ to the voxel Vᵢ in the synthesized mesh.

10. The method as claimed in claim 9, further comprising integrating a mask containing geometric features in high contrast.

11. The method as claimed in claim 1, wherein said developing 3D synthesis comprises
for each voxel in a synthesized mesh wherein an input is a cubic region Regᵢ from the synthesized mesh that defines a hole, centered at Vᵢ.:
getting a cubic region Regⱼ from the sample mesh, centered at Vⱼ;
defining a measure distance dⱼⱼ, between Regᵢ and Regⱼ, such that dᵢⱼ=d(Regᵢ, Regⱼ);
creating a set {Regⱼ} having good correlation with Regᵢ;
selecting Regⱼ with the highest correlation from the set {Regᵢ};
copying {Regⱼ} to the synthesized mesh.

12. The method as claimed in claim 1, further comprising adding parameters characterizing the bone.

13. The method as claimed in claim 12, wherein said parameters are selected from a group of parameters such as bone density and directionality.

14. A reconstructed implant to be filled within a hole in a fractured or diseased bone that is designed according to the method claimed in claim 1.

15. The reconstructed implant as claimed in claim 14, wherein said implant fills in the hole in a seamless manner.
